(19) United States
(12) Patent Application Publication
(10) Pub. No.: US 2010/0256773 A1
(43) Pub. Date: Oct. 7, 2010

(54) SURGICAL IMPLANT COMPOSED OF A POROUS CORE AND A DENSE SURFACE LAYER

(75) Inventors: Ivo Thijs, Mol (BE); Wim Bouwen, Westerlo (BE); Steven Mullens, Zichem (BE); Jan Luyten, Molenstede (BE); Norbert Dejonghe, Sint Andries (BE)

Correspondence Address: MERCHANT & GOULD PC, P.O. BOX 2903, MINNEAPOLIS, MN, 55402-0903, US

(73) Assignee: Vlaamse Instelling Voor Technologisch Onderzoek N.V. (VITO), Mol (BE)

(21) Appl. No.: 12/666,156

(22) PCT Filed: Jul. 3, 2008
(86) PCT No.: PCT/EP08/58625

§ 371 Date: Jun. 7, 2010

(30) Foreign Application Priority Data
Jul. 3, 2007 (EP) 0711662.8

(51) Int. Cl. A61F 2/28; B05D 3/02

(52) U.S. Cl. 623/23.55; 427/2.24

(57) ABSTRACT

A surgical implant (10) includes a porous core part (11) made of a porous biocompatible material and a dense shell (12) made of a biocompatible material provided on a part of the surface of the porous core part which forms an interface with biological soft tissue. The dense shell shields the porous core from in-growth of soft tissue. The porous core part has open interconnected pores. A method of manufacturing a surgical implant includes the steps of: producing a porous core part, applying a viscous suspension on a part of the surface of the porous core part and applying a thermal treatment.

### FIELD OF THE INVENTION

The present invention is related to a surgical implant and a method of manufacturing thereof. Particularly, the invention is related to a bone implant and a method of manufacturing thereof.

### STATE OF THE ART

Titanium and Ti-alloys are commonly used for the production of surgical implants, because of their strength/weight ratio, their high resistance to corrosion and relatively low elasticity modulus, and their biocompatibility. Therefore, Ti and Ti-alloys are already used in a large number of implant materials in a dense form, e.g. for dental implants, shoulder-, hip-, finger-implants, etc.

Dense titanium implant materials have the disadvantage to loose their fixation after some time due to their higher strength and higher E-modulus compared to trabecular bone. This phenomenon is known as “stress shielding” [1,2]. A conventional way of improving the fixation is to increase the roughness of the surface or to coat it with a calcium phosphate layer.

Nevertheless, many implant materials are loosing their fixation with the bone by “stress shielding” with extra painful operations and high costs as a result.

### REFERENCES

1. Garrett Ryan, Abhay Pandit, Dimitrios Panagiotis Apatsidis, “Fabrication methods of porous metals for use in orthopaedic applications”, Biomaterials 27 (2006), pp. 2651-2670
2. P. Habibovic, J. Li, C. M. vanderValk, G. Meijer, P. Layrolle, C.A. Van Blitterswijk, K. de Groot, Biomaterials 26 (2005), pp. 23-36

### AIMS OF THE INVENTION

The present invention aims to provide a surgical implant which overcomes the drawbacks of prior art implants. It is an aim of the invention to provide at least an alternative surgical implant with at least the same and preferably an improved functionality compared with prior art implants. The present invention equally aims to provide a method of manufacturing the implant.

### SUMMARY OF THE INVENTION

The present invention is related to an improved surgical implant and to a method of manufacturing said implant, as set out in the appended claims. The surgical implant of the invention comprises (or consists of) a porous core part made of a porous biocompatible material and a dense shell made of a biocompatible material. The dense shell is provided on a part of the (outer) surface of the porous core part. The dense shell is provided for preventing in-growth of biological soft tissue into the porous core part.

Biological soft tissue refers to tissues that connect, support, or surround other structures and organs of the body, without being bone tissue. Soft tissue comprises muscles, tendons, fibrous tissues, fat, blood vessels, nerves, and synovial tissues. In the present disclosure, biological soft tissue does not have the meaning of bone tissue.

Preferably, the dense shell is arranged to be completely covered by biological soft tissue. The surgical implant of the invention is arranged for being implanted onto the bone and under the skin. The porous core part is arranged to be in contact with bone. The dense shell shields the porous core part from penetration of soft tissue into the pores of the porous core part. Therefore, the dense shell is provided on that part of the surface of the porous core which forms an interface between the porous core and the soft tissue (i.e. that part of the surface of the porous core which is not covered by bone at the time of implant). This allows bone tissue to grow into the porous core part without being hindered by excessive in-growth of soft tissue into the porous core part. Preferably, the dense shell is not provided at the interface between bone and the porous core part.

The surgical implant of the invention hence comprises (or consists of) a porous part and a dense shell. The porous part is made of a porous biocompatible material, preferably a porous metal and the dense shell is made of a dense biocompatible material, preferably a metal. More preferably, the porous core part is made of a porous titanium or a porous Ti-alloy. More preferably, the dense shell is made of titanium or Ti-alloy. The porous part and the dense shell are bonded to each other. The dense shell is impenetrable for biological tissue.

Preferably, the porous core part is made of a porous ceramic material. More preferably, the ceramic material is a calcium phosphate. The ceramic material is advantageously: hydroxyapatite, alpha-tricalcium phosphate, or beta-tricalcium phosphate. The ceramic material can advantageously be a combination thereof, in particular as a biphasic or triphasic calcium phosphate. The ceramic material can be a SiO₂ substituted calcium phosphate. The ceramic material can be an oxide, preferably of aluminium or zirconia.

Preferably, the dense shell is made of a ceramic material as indicated hereinabove.

Preferably, the dense shell has a thickness falling in the range between 200 μm and 1000 μm, more preferably falling in the range between 300 μm and 500 μm.

Preferably, the porous core part comprises open interconnected pores. The pores of the porous core part are preferably tortuous. The pores are preferably permeable to bone cells. The pores have preferably a size in the range between 50 μm and 1500 μm, more preferably in the range between 50 μm and 1000 μm, most preferably in the range between 50 μm and 500 μm. The mean size of the pores of the porous core part lies preferably in the range between 100 μm and 500 μm.

Preferably, the porous core part has a porosity falling in the range between 25% and 95% of the theoretical density (i.e. a density between respectively 75% and 5% TD). Even more preferably, said porosity falls in the range between 60% and 90% theoretical density. Particularly preferably, said porosity falls in the range between 70% and 80% theoretical density.

Preferably, the surgical implant according to the invention further comprises plates or strips. The plates or strips are preferably provided with openings for attachment of the surgical implant to the bone.

Advantageously, the surgical implant according to the invention may comprise means for fastening one or more dental prostheses (dental crowns). Said means for fastening a dental prosthesis are preferably one or more dental implants or tooth roots.

According to a second aspect of the invention, there is provided a method of manufacturing a surgical implant of the invention. The method comprises the steps of: producing a porous core part of a first biocompatible material, applying a suspension on at least a part of the surface of the porous core part to obtain a coated core part and applying a thermal treatment to the coated core part. The suspension preferably comprises a powder of the first biocompatible material. The suspension can comprise a powder of a second biocompatible material.

Preferably, the first biocompatible material is titanium or a Ti-alloy and the second biocompatible material is titanium or a Ti-alloy.

Preferably, the step of producing a porous core part comprises using a gelcasting technique. Equally preferably, the step of producing a porous core part comprises using a 3D fibre deposition technique. A rapid prototyping technique can be used as well for producing the porous core part.

Preferably, the step of applying a suspension comprises painting or brushing the suspension on a part of the surface of the porous core part. The step of applying a suspension can comprise spraying the suspension on a part of the surface of the porous core part. Equally preferably, the step of applying a suspension comprises tape-casting the suspension on a part of the surface of the porous core part.

Preferably, the step of applying a suspension consists of applying a suspension on that part of the surface of the porous core part which is configured to be in contact with (or form an interface with) biological soft tissue for preventing in-growth of said biological soft tissue into the porous core part. More preferably, prior to the step of applying the suspension, methods of the invention comprise a step of determining that part of the surface of the porous core part which is configured to be in contact with (or form an interface with) biological soft tissue. In the determining step, said part of the surface of the porous core part is determined, which is configured to be shielded from penetration by biological soft tissue.

Preferably, the step of applying a thermal treatment comprises the step of sintering the coated core part. The thermal treatment can comprise a pre-sintering step. The thermal treatment more preferably comprises a calcining step.

### BRIEF DESCRIPTION OF THE DRAWINGS

FIG. 1 represents a cross-section of a surgical implant of the invention, comprising a porous core and partially surrounded by a dense shell.

FIG. 2 represents the implant of FIG. 1 implanted in bone.

FIG. 3 represents a design of a surgical implant according to the invention provided with holes for accepting dental implants.

FIG. 4 represents the surgical implant of FIG. 3 provided with three dental implants and implanted in bone.

FIG. 5 represents a porous core structure provided with a dense shell according to the invention.

### DETAILED DESCRIPTION OF THE INVENTION

Embodiments of the present invention will now be described in detail with reference to the attached figures, the invention is not limited thereto but only by the claims. The drawings described are only schematic and are non-limiting. In the drawings, the size of some of the elements may be exaggerated and not drawn on scale for illustrative purposes. The dimensions and the relative dimensions do not necessarily correspond to actual reductions to practice of the invention. Those skilled in the art can recognize numerous variations and modifications of this invention that are encompassed by its scope. Accordingly, the description of preferred embodiments should not be deemed to limit the scope of the present invention.

Furthermore, the terms first, second and the like in the description and in the claims are used for distinguishing between similar elements and not necessarily for describing a sequential or chronological order. It is to be understood that the terms so used are interchangeable under appropriate circumstances and that embodiments of the invention described herein are capable of operation in other sequences than described or illustrated herein.

Moreover, the terms top, bottom, left, right, over, under and the like in the description and the claims are used for descriptive purposes and not necessarily for describing relative positions. The terms so used are interchangeable under appropriate circumstances and embodiments of the invention described herein can operate in other orientations than described or illustrated herein. For example, “left” and “right” of an element indicates being located at opposite sides of this element.

It is to be noticed that the term “comprising”, used in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, A and B are relevant components of the device.

Where numerical values are given with regard to limitations of a quantity, or the outcome of a measurement, for the assessment of those values, account shall be taken of variations due to impurities, methods used to determine measurements, human error, statistical variance, etc.

Where a range of numerical values is defined as extending between a lower limit and an upper limit, the range is to be construed as including said lower limit and said upper limit, unless otherwise noted.

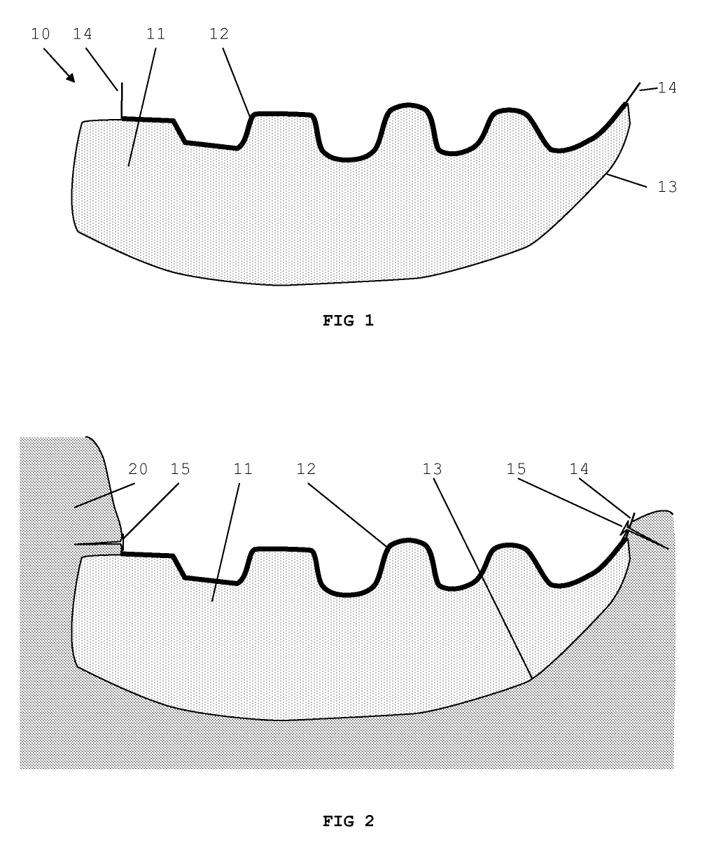

FIG. 1 shows a cross-section of a surgical implant 10 according to the invention. Surgical implant 10 may be a bone implant for filling a cavity or replacing a damaged bone structure. Other shapes for the implant are equally envisaged by the present invention and the implant of FIG. 1 is merely used for illustrative and descriptive purposes.

Surgical implant 10 comprises a core 11 of a porous biocompatible material (e.g. titanium or a Ti-alloy). On a part of the surface of the porous core part 11 a dense shell 12 of a biocompatible material (e.g. titanium or a Ti-alloy) is provided. An other part 13 of the surface of the porous core is not covered by the shell 12. At a few locations on the surface of the implant small plates 14 are attached to the porous core part 11 or to the dense shell 12. These plates are provided with holes for passing screws through them in order to fasten the implant onto the bone.

The porous core 11 is preferably made of a (porous) biocompatible metal, preferably titanium or a Ti-alloy, in particular an alloy with aluminium and vanadium (e.g. Ti-6Al-4V). Other metals that can alternatively be used are stainless steel and alloys of cobalt, chromium and molybdenum (Co—Cr—Mo alloys).

Alternatively, the porous core 11 is equally preferably made of a (porous) biocompatible ceramic material. The ceramic material is preferably a calcium phosphate, such as hydroxyapatite, alpha-tricalcium phosphate, beta-tricalcium phosphate, or a combination thereof (bi- or tri-phasic calcium phosphates). The ceramic material can be a SiO₂ substituted calcium phosphate. The ceramic material can be a ceramic oxide, such as aluminium oxide or zirconia oxide.

The dense shell 12 can be made of a biocompatible material as indicated hereinabove (for the porous core 11). The dense shell 12 can be made of a material different from the material of the porous core 11. Preferably, dense shell and porous core are made of a same material. This is advantageous for preventing disparate thermal expansion coefficients.

FIG. 2 shows the surgical implant 10 implanted in bone 20. The implanting in the bone 20 is such that the porous core part 11 is in direct contact with the bone, with the another part 13 of the surface of porous core part 11 forming the interface between bone 20 and implant 10. The implant is fastened to the bone with screws 15 passing through the plates 14.

The core part 11 is permeable to bone cells. The pores of core part 11 should have a size so as to allow cell growth within the porous structure of core part 11. The growth of bone cells within a porous structure of a biocompatible material has been found to occur for pores having a size of 50 μm and more. Preferably, the pores of the core 11 are tortuous and are open interconnected pores.

The pore size distribution of the porous core part 11 can be customized. The pore size of the porous core part 11 preferably lies in the range between 50 μm and 1500 μm, more preferably in the range between 50 μm and 1000 μm and most preferably in the range between 50 μm and 500 μm. Pore size can be measured by image analysis.

The mean pore size of the porous core part preferably falls in the range between 100 μm and 500 μm, which is generally recognized as ideal for allowing bone in-growth. More preferably, the mean pore size of the porous core part falls in the range between 200 μm and 400 μm, most preferably in the range between 200 μm and 300 μm.

The porosity of the core 11 may range between 25% and 95% of the theoretical density (TD), with the range between 60% and 90% TD being preferred and the range between 70% and 80% TD being more preferred. Porosity ranges as indicated constitute an optimal compromise between open structure and mechanical strength of the core 11. Hence, bone can grow into the porous structures of the core 11, improving the fixation of the implant. The enhanced bone in-growth creates a biological fixation and minimizes the problem of stress shielding. The appropriate choice of material for the porous core and of the amount of porosity allows to obtain a porous core having mechanical properties comparable to that of the surrounding bone.

The material and porosity of the porous core part 11 are preferably so chosen that the compressive strength of the porous core part 11 is at least 40 MPa, more preferably between 40 MPa and 75 MPa and particularly preferably between 50 MPa and 75 MPa.

The porous core 11 hence allows bone in-growth, which allows the damaged osseous structure to restore well. However, the porous core is not only permeable to bone tissue, but also to the soft tissue that surrounds the bone structure. Mostly, the soft tissue shows even higher in-growth rates than the bone tissue. As a result, the in-growth of bone tissue is hindered, with an incomplete healing of the damaged structure as a result. In order to overcome this problem, the surgical implant of the present invention comprises a dense shell 12, whose function is to shield the porous core 11 from soft tissue surrounding the bone 20.

The term “dense” is defined here as impenetrable for biological tissue. A clear advantage of the dense shell is that it can shield the porous core from in-growth of soft tissue surrounding the bone.

Pore sizes smaller than 10 μm, preferably smaller than 2 μm were found impenetrable for biological tissue.

The dense shell 12 should be provided on that part of the surface of the porous core 11 which will be in contact with the soft tissue. It should not be provided on the part of the surface of the porous core which forms the interface with the bone structure. It should be noted that along the border between the implant and the bone structure, small gaps between the dense shell and the bone are tolerable. Soft tissue is likely to grow into the porous core at these spots, but as long as these areas remain small, there is no relevant effect to the whole of the implant. It is preferable though that the dense shell closes the border between bone and implant.

The outer surface of the dense shell 12 has preferably a low roughness, so as to prevent soft tissue from adhering to the dense shell 12. The roughness of the outer surface of the dense shell is preferably smaller than or equal to 1 μm Ra, more preferably smaller than or equal to 0.7 μm Ra.

The thickness of the dense shell 12 lies in the range between 200 and 1000 μm, preferably between 300 and 500 μm. The dense shell may additionally increase the strength of the implant.

The dense shell 12 is preferably (additionally) used for aesthetical reasons (e.g. the flatness for facial components). The dense shell 12 can prevent that the skin covering a bone reconstruction by a surgical implant 10 gets wrinkled by excessive ingrowth of the skin tissue in the porous structure of the implant. Such wrinkling of the skin would severely deteriorate the aesthetical aspect of e.g. a facial reconstruction.

Surgical implants of the invention are preferably used for reconstruction of parts of facial bones, such as the jaw. Surgical implants of the invention are advantageously used for reconstruction of cranial bone. Surgical implants of the invention can be used for bone reconstruction in animals, such as dogs, horses, etc.

A second aspect of the invention is related to a method of manufacturing the surgical implant of the invention.

In a step of the manufacturing method, the porous core part 11 is produced. Porous core parts are preferably produced by a foam technique. They can alternatively be produced by a 3D fibre deposition technique. They can be produced by a rapid prototyping technique. For example, these techniques allow to manufacture porous titanium or Ti-alloy. Reference is made here to patent application WO 2006/130935, describing the use of gel-casting to produce porous bodies in titanium or a Ti-alloy, and to the paper “Porous Ti6Al4V scaffold directly fabricating by rapid prototyping: preparation and in vitro experiment”, Jia Ping Li et al., Biomaterials 27 (2006) pp. 1223-1235, describing the use of rapid prototyping for that purpose.

The porous core part is preferably produced by gel-casting.

Careful control of the production parameters of the porous core allows to obtain a wide variety in (micro)structural characteristics (porosity, pore size distribution, etc.) of the porous core. These production process parameters may be chosen out of the group comprising: powder quality, viscosity of the suspension, loading, composition, gelling agent and concentration thereof, foaming agent, mixing time, calcination parameters (temperature, heating rate, vacuum pressure and time period), sintering parameters (temperature, heating rate, vacuum pressure and time period), etc. Hence, the mechanical properties and the porosity of the porous core can be tuned within a wide range, avoiding a large discrepancy in mechanical properties of the porous core with these of the surrounding bone. At this stage of the manufacturing procedure, the porous core part 11 may be in green (not sintered), pre-sintered (having undergone a thermal treatment at e.g. 1000° C., but not sintered), or sintered state.

In a following step, a suspension is provided on a part of the surface of the porous core part 11. The part of the surface of porous core 11 that has to be shielded is determined and on that part a superficial layer of the porous core is locally densified with a suspension comprising a powder of a biocompatible material. The suspension has preferably a high viscosity so as to avoid excessive permeation of the suspension into the pores of the porous core. The suspension may be painted, sprayed or brushed on the surface of the porous core part. The suspension may equally be applied on the porous core part by tape casting, or by other techniques known in the art. The application of the suspension on the surface of the porous core part results in the closure of the surface pores.

Determining the part of the surface of porous core 11 that has to be shielded (by a dense shell) can be performed by virtual (computer aided) reconstruction of the damaged bone, such as is known in the art. The determining step can be carried out prior to the production of the porous core part 11. Computer aided drawing programs allow to design the form and shape of the surgical implant. They also allow to determine the part of the surface of the porous core 11 which does not form an interface with the bone. That part, which would form an interface with biological soft tissue, is envisaged to be covered by a suspension.

In a subsequent step, the porous core part 11 and the applied suspension is subjected to a thermal treatment in order to convert the suspension to a dense, solid shell. The thermal treatment may comprise a sintering step. In the case that the suspension is applied on a green porous core, the sintering step is preceded by a calcination step, preferably at temperatures between 400° C. and 600° C. A slow heating until temperatures as indicated is preferred, such as with a heating rate smaller than or equal to 25° C. per hour, more preferably smaller than or equal to 20° C. per hour. Calcination is preferably performed in vacuum (at pressures around 10⁻³ mbar, preferably between 10⁻⁵ mbar and 10⁻³ mbar) and/or in an argon atmosphere. In the calcination step, organic components which are still present in the green body are burnt out. A burning out of the organic components during sintering would otherwise produce gasses which would induce cracks and damage the structure.

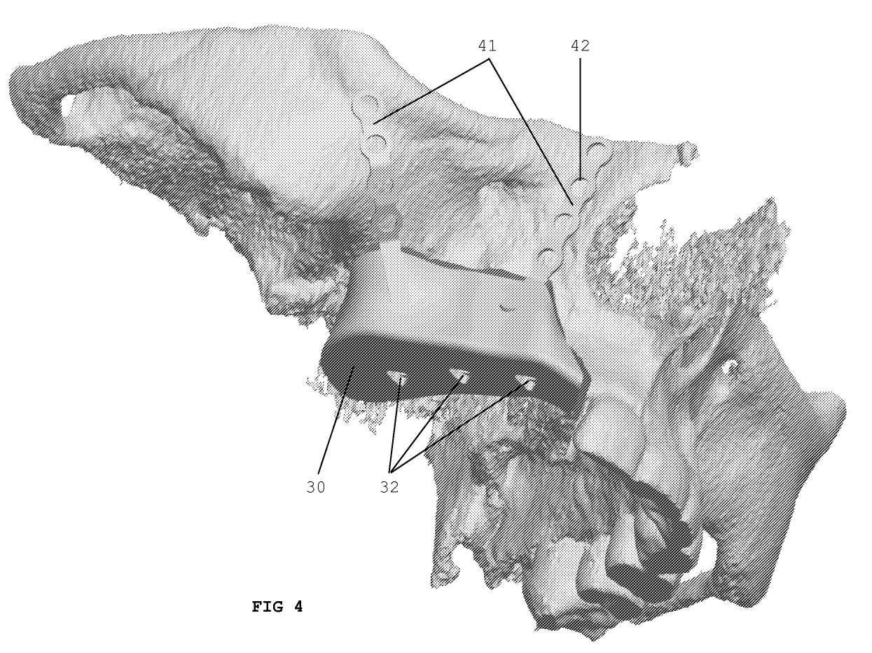

In an optional step, plates or strips 14 (plates 41 in FIG. 4) provided with holes (holes 42 in FIG. 4) may be attached to the porous core part 11 for fixing the implant to the bone. This step may be performed before or after the application of the dense shell. The implant is usually fastened by screws passing through the holes of the plates 14. The plates may be attached to the implant 10 by laser welding, sintering or any other technique known in the art.

According to a particular embodiment of the invention, titanium or a titanium alloy are used as materials for the dense shell and porous titanium or a porous titanium alloy are used as materials for the porous core. The suspension for the dense shell in that case comprises a powder of Ti or a Ti-alloy.

Alternatively, other biocompatible metals, or even ceramic materials as indicated above can advantageously be used as materials for the dense shell and/or porous core in methods of the invention.

Conventional machining techniques may be used to give the porous core the right dimension or to bring the combined component (porous core and dense shell) after the thermal treatment step to the specified tolerances. The dense shell may be ground, polished, etc. to obtain a low surface roughness.

The co-sintering of dense and porous Ti or Ti-alloy parts creates the possibility to develop a large number of improved medical components.

### DESCRIPTION OF A PREFERRED EMBODIMENT OF THE INVENTION

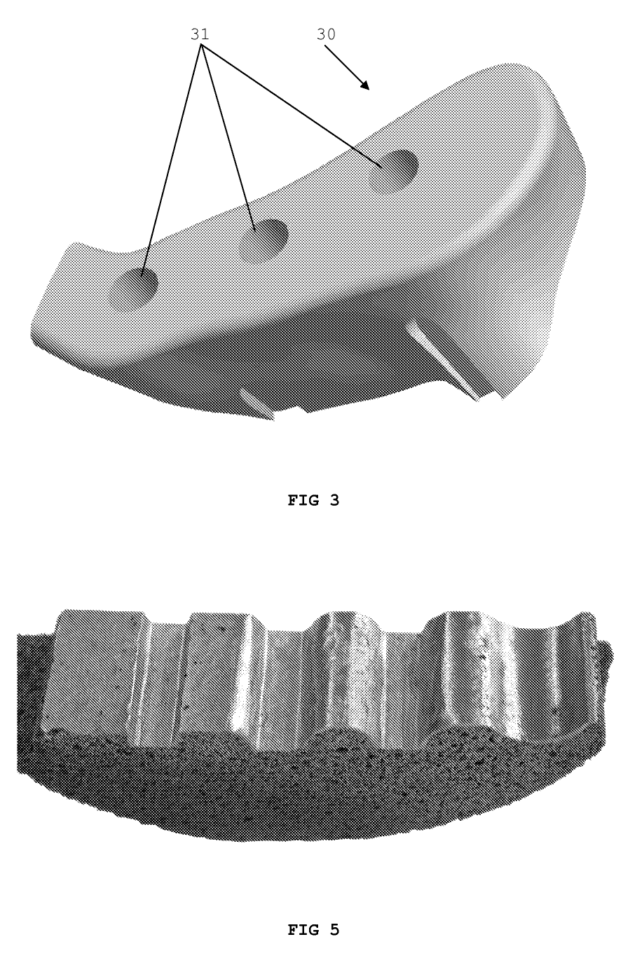

For replacing part of the jaw-bone, or filling a cranium cavity, caused by an accident or operation, a porous titanium, or Ti-alloy, implant material is preferably used. An example of such an implant design is given in FIG. 3. FIG. 4 shows the implant 30 of FIG. 3 implanted in the bone structure.

For each patient a customized implant design has to be made starting from medical scans. Therefore FIG. 3 only exemplifies the principle of design.

The porous core part is made of Ti, Ti6Al-4V or another Ti-alloy and is manufactured by gelcasting. A suspension is prepared using 300 g Ti (T-1147, Cerac, −325 mesh), 201 g H₂O, 6 g Agar (3.18% on H2O), 6 g Tergitol TMN10 (2% on Ti), 3 g Triton (1% on Ti) and 0.36 g ammonium alginate (0.18% on H2O). The suspension is mixed during 6 minutes at 70° C. to obtain a fluid foam. The foam is cast into a mould and cooled down until the structure is gellified. After demoulding, the structure is dried at atmospheric pressure and room temperature. The structure is calcined (10⁻³ mbar, 25° C./hour until 500° C.), pre-sintered at 1000° C. during 2 hours isothermally and sintered at 1350° C. in a vacuum of 10⁻⁵ mbar. The heating from 1000° C. to 1350° C. was done at 5° C./minute.

The porous core part thus obtained having pore sizes in the range between 100 μm and 500 μm with a mean pore size of 300 μm, is machined to the specified dimensions. For fixing the implant to the residual jaw-bone, small plates 41 are sintered by laser welding under protected atmosphere to the porous core part. These plates 41 have openings 42 for fixing the implant to the bone with the aid of screws.

In order to avoid soft tissue in-growth into the porous core part of the implant, a dense Ti shell is provided at places which are envisaged to be in contact with the soft tissue (i.e. that part of the surface of the porous core that, at time of implant, is not in contact with bone). Hence, a Ti-suspension is applied onto the surface of the porous core part, at places where the dense shell is to be foreseen. A possible composition for the Ti-suspension is as follows:

50 g Ti powder (Cerac T-1147),

### 14.83 g H₂O,

### 0.03 g Benecel,

0.667 g gelatin,
0.85 g targon 1128,
0.15 g antifoam.
This viscous suspension is painted, sprayed or brushed on the surface of the porous core part, resulting in the closure of the surface pores.

Next, the part is calcined (at 500° C. in vacuum of 10⁻³ mbar for at least one hour) and sintered (at 1350° C. in vacuum of 10⁻⁵ mbar for 2 hours).

After these thermal treatments, the dense shell can be further polished to a desired roughness. FIG. 5 shows an example of a porous structure which is provided with such a dense shell.

In the case that the implant is used to replace part of the jaw bone, surgical implant 30 allows to incorporate one or more dental implants (tooth roots) 32. As shown in FIG. 3, holes 31 may be provided in the porous core of the surgical implant 30. These holes accept dental implants, such as the ones that are known in the art. The dental implants are bonded to the surgical implant 30, e.g. by sintering or any other technique known in the art.

In the case that the dental implant and the surgical implant are made of Ti or a Ti-alloy, they may be bonded by sintering. The bonding between surgical and dental implant may be improved by adding fine sinter-active powder (e.g. Ti-powder in case of Ti or Ti-alloys) on one or both surfaces that are brought into contact, or by sandblasting the surfaces in order to remove the TiO₂ layer. The combined bone and dental implant hence obtained allows a person who has lost part of the jaw and corresponding teeth to regain both the functionalities of the lost teeth and the jaw bone in a reduced time span.

While the foregoing description and drawings represent the preferred embodiments of the present invention, it will be obvious to those skilled in the art that various changes and modifications may be made therein without departing from the spirit and scope of the present invention.

1. A surgical implant comprising a porous core part made of a porous biocompatible material and a dense shell made of a biocompatible material, wherein the shell is provided on that part of the surface of the porous core part which is configured to form an interface between the porous core part and biological soft tissue and for preventing in-growth of said biological soft tissue into the porous core part.

2. The surgical implant according to claim 1, wherein the dense shell has a thickness in the range between 200 μm and 1000 μm.

3. The surgical implant according to claim 1, wherein the porous core part is made of porous titanium or a porous Ti-alloy and the dense shell is made of titanium or a Ti-alloy.

4. The surgical implant according to claim 1, wherein the porous core part is made of a porous ceramic material and the dense shell is made of a ceramic material.

5. The surgical implant according to claim 1, wherein the porous core part comprises open interconnected pores.

6. The surgical implant according to claim 1, wherein the porous core has a porosity falling in the range between 25% and 95% theoretical density.

7. The surgical implant according to claim 1, comprising plates or strips provided with openings for attachment of the surgical implant to the bone.

8. The surgical implant according to claim 1, further comprising means for fastening one or more dental crowns.

9. A method of manufacturing a surgical implant comprising the steps of:
producing a porous core part of a first biocompatible material,
applying a suspension comprising a powder of the first biocompatible material or of a second biocompatible material on at least a part of the surface of the porous core part to obtain a coated core part and
applying a thermal treatment to the coated core part.

10. The method according to claim 9, wherein the first biocompatible material is titanium or a Ti-alloy and the second biocompatible material is titanium or a Ti-alloy.

11. The method according to claim 9, wherein the step of producing a porous core part comprises using a gelcasting technique, 3D fibre deposition technique or a rapid prototyping technique to produce the porous core part.

12. The method according to claim 9, further comprising the step of determining said part of the surface of the porous core part prior to the step of applying a suspension, said part of the surface configured to being shielded from penetration by biological soft tissue.

13. The method according to claim 11, wherein the step of applying a suspension comprises painting, spraying, brushing or tape-casting the suspension on a part of the surface of the porous core part.

14. The surgical implant according to claim 1, wherein the dense shell has a thickness in the range between 300 μm and 500 μm.

15. The surgical implant according to claim 1, wherein the porous core part is made of a calcium phosphate and the dense shell is made of a calcium phosphate.

16. The surgical implant according to claim 1, wherein the porous core has a porosity falling in the range between 60% and 90% theoretical density.
